(19) United States
(12) Patent Application Publication
(10) Pub. No.: US 2007/0213802 A1
(43) Pub. Date: Sep. 13, 2007

(54) Bifurcation Stent Delivery Catheter and Method

(75) Inventors: Randolf Von Oepen, Los Altos Hills, CA (US); Thomas Rieth, Hirrlingen (DE); Lorcan Coffey, Tubingen (DE); Richard Newhauser, San Francisco, CA (US); Travis Yribarren, San Mateo, CA (US)

Correspondence Address: SQUIRE, SANDERS & DEMPSEY L.L.P., 1 MARITIME PLAZA, SUITE 300, SAN FRANCISCO, CA, 94111, US

(73) Assignee: Abbott Laboratories, Redwood City, CA (US)

(21) Appl. No.: 11/683,899

(22) Filed: Mar. 8, 2007

Related U.S. Application Data
(60) Provisional application No. 60/780,135, filed on Mar. 8, 2006.

(51) Int. Cl. A61F 2/84

(52) U.S. Cl. 623/1.11

(57) ABSTRACT

A stent delivery system is disclosed for delivering and deploying a radially expandable stent at a strategic orientation and location in a body vessel. The delivery system includes an elongated flexible tubular shaft sized suitably for insertion into the body vessel, first and second inflatable members disposed adjacent the distal end of the elongated shaft and an endoprosthesis disposed about the first and second inflatable members. The delivery system further includes a tip assembly which during to advancement of the delivery system is configured as a single tip assembly, wherein prior to deployment of the expandable endoprosthesis, the tip assembly is split into a first tip and a second tip, wherein one of the tips remains in a main branch and the second tip is advanced into a side branch lumen to align the endoprosthesis prior to deployment.

### FIELD OF THE INVENTION

The present invention relates generally to catheters and systems used for delivering devices such as, but not limited to, intravascular stents and therapeutic agents to sites within vascular or tubular channel systems of the body. More particularly, it relates to delivery catheters and systems for delivering stents to bifurcated vessels.

### BACKGROUND OF THE INVENTION

A type of endoprosthesis device, commonly referred to as a stent, may be placed or implanted within a vein, artery or other tubular body organ for treating occlusions, stenoses, aneurysms or dissections of a vessel by reinforcing the wall of the vessel or by expanding the vessel. Stents are normally placed to scaffold the vessel and avoid elastic recoil after angioplasty. Another reason for applying stent is it to treat dissections in blood vessel walls caused by balloon angioplasty of the coronary arteries as well as peripheral arteries and to improve angioplasty results by preventing elastic recoil and remodeling of the vessel wall. Two randomized multicenter trials have shown a lower restenosis rate in stent treated coronary arteries compared with balloon angioplasty alone (Serruys, P W et al. New England Journal of Medicine 331: 489-495, 1994, Fischman, D L et al. New England Journal of Medicine 331:496-501, 1994). Stents have been successfully implanted in the urinary tract, the bile duct, the esophagus and the tracheo-bronchial tree to reinforce those body organs, as well as implanted into the neurovascular, peripheral vascular, coronary, cardiac, and renal systems, among others. The term “stent” as used in this Application is a device that is intraluminally implanted within bodily vessels to reinforce collapsing, dissected, partially occluded, weakened, diseased or abnormally dilated or small segments of a vessel wall.

One common procedure for intraluminally implanting a stent within a body vessel is to first dilate the relevant region of the vessel with a balloon catheter. Subsequently, a delivery catheter, such as Percutaneous Transluminal Coronary Angioplasty (PTCA) Catheters containing a dilator at the distal end thereof, is applied to transport a stent to the lesion site, and to deploy the stent in a position that bridges the affected portion of the vessel. The expanded stent provides scaffolding to the lumen that allows adequate blood flow within the lumen. These delivery catheters typically include a relatively long flexible shaft (e.g., normally about 145 cm in length that is sized to be percutaneously inserted into the vessels) with a dilator or stent deployment assembly at the distal end of the shaft that carries the stent.

During any such catheterization and interventional procedures, including for example angioplasty and/or stenting, a hollow needle is initially applied through a patient's skin and tissue to facilitate advancement of the catheter shaft through the target vasculature. As is often the case, however, the catheter shaft may need to be inserted into vessels having a relatively tortuous path leading to the lesion site. Since it can be difficult to steer many types of catheters, guidewires are applied to facilitate advancement of the catheters through the vessel. Guidewires are typically formed from a very small diameter metallic wire having a flexible tip that can be rotatably controlled to some degree. The operator is shaping the tip of the guidewire by bending it depending on the anatomy of the vessel. Since the guidewire body is transmitting torque very well, the tip of the catheter can be steered through the anatomy of the patient. Furthermore steerable guidewires have been developed which allow the operator to deflect the tip of the wire actively in the vasculture of the patient. The ability to rotatably control the tip is important in that the guidewire can be steered to access a desired location through a potentially tortuous path such as the vasculature.

Once the guidewire is advanced through the needle and into the patient's blood vessel, the needle is removed. An introducer sheath is then advanced over the guidewire into the vessel, e.g., in conjunction with or subsequent to a dilator. The catheter or other deployment device may then be advanced through a lumen of the introducer sheath and over the guidewire into a position for performing a medical procedure. Thus, the introducer sheath may facilitate introducing various devices into the vessel, while minimizing trauma to the vessel wall and/or minimizing blood loss during a procedure.

In some applications, the targeted region of a vessel may be at a location where the vessel bifurcates. For example, in cases where plaque has developed in the region of a vessel bifurcation, it may be desirable to perform angioplasty, atherectomy, and/or stenting in one or all of the affected vessels. In general, it is very important to preserve the side branch and the main branch of the bifurcation. In some occlusions, it might occur that during the dilation, plaque will be shifted from the treated vessel to the non-treated vessel, and will then occlude that non-vessel. This effect is known as the “snowplow” effect. To enable re-access to the vessel that has been affected by the “snowplow” effect, most physicians prefer to place a guidewire in the non-treated branch as well. If the non-treated vessel is occluded during this procedure, the guidewire positioned in the non-treated vessel will function as a guiding element, and will allow the advance of another catheter to reopen that vessel. In other applications, it may be desirable to insert a bifurcation stent specifically dedicated to treat lesions at a vessel bifurcation.

In the recent past, several commercially available bifurcation stents have been developed that treat bifurcation lesions. By way of example, common alternatives to bifurcation lesion stenting include the Elective T technique, the Provisional T Technique, the Coulotte Technique, the V Technique and the Crush. In addition, dedicated bifurcation systems like the Frontier and AST Systems has been developed. While these bifurcation stent designs have encountered varying degrees of success, one major problem associated with all bifurcation systems is that the delivery and deployment of the stent, relative to the side branch, is extremely difficult. This is due primarily to the difficulty in properly controlling the orientation, alignment and position of the stent deployment assembly relative to the main branch and side branch of the bifurcated vessel.

During advancement of the catheter shaft along the predisposed guidewire, the stent deployment assembly, which supports and transports the stent in a collapsed state, is not rotatably controlled. Hence, it is likely necessary to rotate and reorient the distal delivery assembly about its' longitudinal axis since the bifurcation stent must be properly aligned relative to the side branch before deployment. Current systems that require rotation of the delivery system many times result in less than ideal stent placement.

Other types of delivery systems such as those that utilize two inflatable balloons disposed on the end of a catheter shaft have additional placement difficulties. Many times systems utilizing more than one balloon are tracked over two separate guidewires, wherein one guidewire is placed in the main branch and the second into the bifurcated branch. Problems associated with systems that utilize two separate guidewires have the potential for the two guidewires to become entangled and create difficulties in successful deployment of one or both stents because twists between the guidewires prevent the systems from tracking to the target treatment site, for example.

Accordingly, there is a need for a stent delivery system with improved alignment and orientation capabilities of the distal stent deployment assembly for those stents (e.g., bifurcation stents) that require precise radial alignment relative to the target vessel site.

### SUMMARY OF THE INVENTION

The present invention is directed toward a stent delivery system for delivering and deploying a radially expandable stent at a strategic orientation and location in a body vessel.

In accordance with the present invention there is provided a stent delivery system for delivering and deploying a radially expandable stent at a strategic orientation and location in a body vessel, said delivery system comprising: an elongated shaft; a first inflatable member extending from a distal end of the elongated shaft; a second inflatable member extending from the distal end of the elongated shaft and disposed adjacent to the first inflatable member, wherein the first and second inflatable members are in fluid communication with each other, and a tip assembly, the tip assembly including a first tip portion associated with the first inflatable member and a second tip portion associated with the second inflatable member, wherein the first and second tip portions are configured to be detachably associated with each other.

In accordance with the present invention there is provided a method for placing an expandable endoprosthesis in a lumen, comprising: advancing an endoprosthesis delivery system over at least one guidewire to a location adjacent a bifurcation location, the guidewire extending through a tip assembly; advancing a second guidewire through the tip assembly; separating the tip assembly to form a first tip and a second tip, wherein the first tip is disposed about one of the guidewires and the second tip is disposed about the other guidewire; advancing the delivery system to the bifurcation location; and deploying the endoprosthesis.

In accordance with an alternative embodiment of the present invention there is provided a delivery system configured to deploy an endoprosthesis at a bifurcation, the delivery system comprising

### BRIEF DESCRIPTION OF THE DRAWINGS

The assembly of the present invention has other objects and features of advantage that will be more readily apparent from the following description of the best mode of carrying out the invention and the appended claims, when taken in conjunction with the accompanying drawing, in which:

FIG. 1 is a plan view of an exemplary embodiment of a bifurcation delivery catheter in accordance with the present invention;

FIG. 2 is a plan view of the distal end of the catheter of FIG. 1 illustrating the tip design;

FIG. 3 is an isometric view of an exemplary embodiment of a tip in accordance with the present invention;

FIG. 4 is an isometric view of the tip assembly of FIG. 3 wherein the tip assembly has been separated into a first tip and second tip;

FIGS. 5A through 5D are end views of the tip of FIG. 3, illustrating the separation process of the tip assembly as well as the advancement of the second guidewire in accordance with the present invention;

FIGS. 6A through 6D illustrate the delivery system of the present invention in use;

FIG. 7 is a plan view of an alternative embodiment of a bifurcation delivery catheter in accordance with the present invention;

FIG. 8 is a plan view of the distal end of the catheter of FIG. 7 illustrating the tip design;

FIG. 9 is an isometric view of an alternative embodiment of a tip in accordance with the present invention;

FIG. 10 is an isometric view of the tip assembly of FIG. 9 wherein the tip assembly has been separated into a first tip and second tip; and

FIGS. 11A through 11D illustrate the delivery system of the present invention in use.

### DETAILED DESCRIPTION OF THE PREFERRED EMBODIMENTS

While the present invention will be described with reference to a few specific embodiments, the description is illustrative of the invention and is not to be construed as limiting the invention. Various modifications to the present invention can be made to the preferred embodiments by those skilled in the art without departing from the true spirit and scope of the invention as defined by the appended claims. It will be noted here that for a better understanding, like components are designated by like reference numerals throughout the various figures.

In accordance with the present invention there is provided a catheter, wherein the catheter is configured to deliver and deploy at least one expandable member within a lumen. The catheter includes a first end, a second end and a shaft extending therebetween. At least one inflatable member is disposed adjacent the distal end and coupled to an inflation lumen extending through the shaft of the catheter, preferably at least two inflatable members are disposed adjacent the distal end, wherein each of the inflatable members includes a separate inflation lumen, thereby allowing independent inflation of the inflatable members. In use, the catheter is delivered within a lumen by tracking at least a portion of the catheter over a coaxial guidewire. The distal end is tracked to a location adjacent a bifurcation to be treated. The coaxial guidewire is activated to separate into a main branch guidewire and a side branch guidewire, either simultaneously or in response to the separation of the guidewires the distal tip of the catheter separates from one composite tip to two independent tips

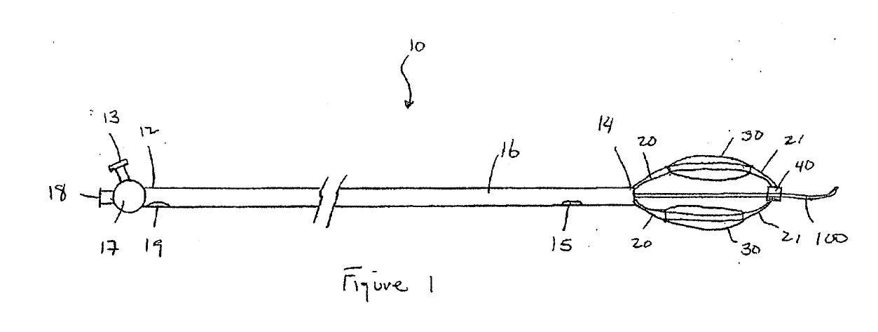

Referring now to FIG. 1 there is shown an exemplary embodiment of the catheter in accordance with the present invention. As shown in FIG. 1, the catheter 10 has a proximal end 12, a distal end 14 and a shaft 16 extending therebetween. A luer fitting 17 may be disposed adjacent the proximal end 12 of the shaft 16. As shown in FIG. 1, the luer fitting includes an aperture 18 disposed therethrough, wherein the aperture 18 is in fluid communication with a lumen 11 (not shown) extending along the length of the shaft 16. Lumen 11 is configured to receive at least one guidewire therethrough as will be described in greater detail below. The luer 17 may further include an inflation fitting 13, wherein the inflation fitting is in fluid communication with at least one inflation lumen 20 (not shown) which extends along the length of the shaft 16, wherein the inflation lumen 20 is in fluid communication with at least one inflatable member 30 disposed adjacent the distal end 14 of the shaft 16 as will be described in detail below.

Disposed adjacent the distal end 14 of the shaft 16 is at least one inflatable member 30 as shown in FIG. 1 and described above. Preferably, the catheter 10 in accordance with an exemplary embodiment of the present invention includes at least two inflatable members 30 disposed adjacent the distal end 14 of the shaft 20. The inflatable members 30 are coupled to the shaft 16 through inflation lumens 20, wherein the inflation lumens 20 extend along the length of the shaft 16 and are fluidly coupled to the inflation port 13 disposed on the luer 17. The inflatable members 30 are in fluid communication with each respective inflation lumen, thereby being capable of being expanded from a first diameter to a second diameter. The distal ends of each of the inflation lumens terminate within the volume of the inflatable member, wherein mandrels 21 extend beyond a distal ends of each of the inflatable members and terminate at a tip member 40. The tip member 40 is comprised of at least two components, wherein the two components interact to form a single tip as shown with a guidewire lumen 41 extending therethrough.

As further shown in FIG. 1, the catheter 10 may be configured to receive a guidewire along its entire length within the guidewire lumen 11 disposed within the shaft 16. Alternatively, as shown in FIG. 1, the catheter 10 may include at least one port 15 formed within the wall of the shaft 16 and in communication with the guidewire lumen 11, wherein a guidewire may be placed within the port 15 and extended beyond the distal end of the shaft 16 as shown. Such a configuration is commonly referred to as a rapid-exchange catheter. It is further contemplated, that the catheter 10 may include a second port 19 adjacent the proximal end 12 of the shaft 16, wherein the second port 19 is associated with the guidewire lumen 11, wherein a guidewire 100 may be disposed thorough the port 19 and along the length of the shaft and through tip 40 of the catheter 10. By allowing the guidewire to pass through the port 19 and not through the aperture 18 of the luer 17, the aperture 18 may be utilized for other functions, such as a second inflation lumen.

Referring now to FIG. 2, there is shown an exemplary embodiment of the distal tip of the catheter 10 in accordance with the present invention, wherein an endoprosthesis such as a stent is shown radially disposed about the first and second inflatable members 30.

Referring now to FIG. 3, there is shown an exemplary embodiment of a tip member 40 in accordance with the present invention. As shown in FIG. 3, the tip member 40 is a composite tip comprising a first tip member 42 and a second tip member 44, wherein when coupled together form the tip member 40 having a lumen 41 extending therethrough.

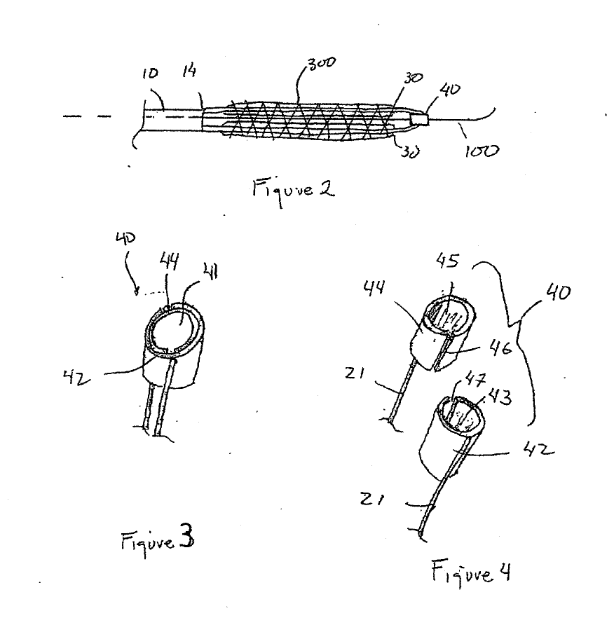

Referring now to FIG. 4 there is shown the tip member 40, wherein the first and second tip members 42 and 44 have been separated into two distinct tip members each having a lumen 45 and 43 disposed therethrough respectively. Each of the first and second tip members are fixedly attached to the distal mandrels, 21, wherein when separated as shown in FIG. 4, the catheter 10 converts from a single tipped catheter to a dual tip catheter as will be described below with regard to the methods in accordance with the present invention. The bodies of the tip members 42 and 44 are preferably constructed having a generally cylindrical profile, wherein a slot is disposed extending along a longitudinal length of the bodies, wherein the slot enables the bodies to expand in diameter, thereby allowing the bodies of the tip members 42 and 44 to nest together as shown in FIG. 3. The tip members 42 and 44 are preferably constructed of a resilient pliable material, wherein the bodies are capable of flexing and/or be expanded about one another to nest together as shown in FIG. 3. Additionally, the tip members 42 and 44 must be constructed of a material which has a sufficiently low durometer such as to not cause damage to a lumen in which the catheter is disposed therein.

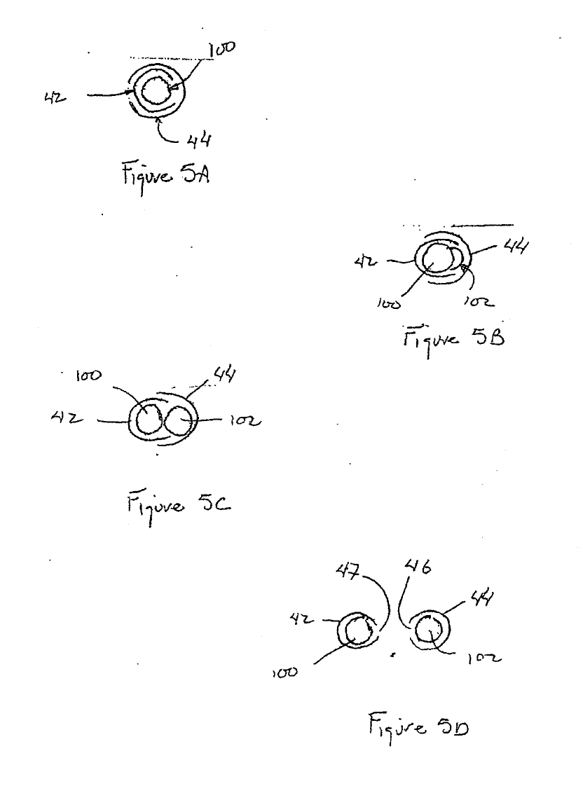

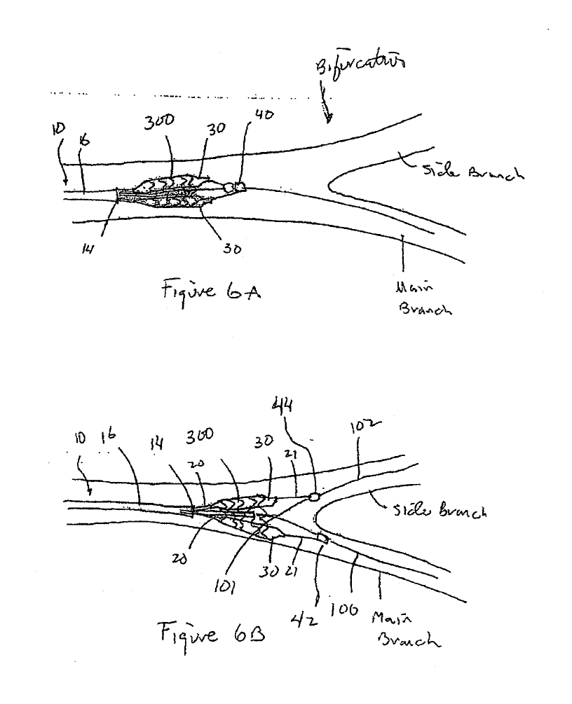

Referring now to FIGS. 5A through SD there is shown the sequence of tip separation that occurs prior to placement of the distal tip of the catheter in a desired location. Referring now to FIG. 5, there is shown a cross-sectional view of the composite tip assembly 40 in accordance with the present invention. As show, the composite tip assembly 40 comprises a first tip member 42 and a second tip member 44, wherein the first tip member 42 is radially received within a lumen of the second tip member 44. Additionally as shown, a guidewire is shown disposed through the aperture 41 of the tip assembly 40.

Referring now to FIG. 5B there is shown a cross-sectional view of the composite tip assembly 40 in accordance with the present invention wherein the two tip portions 42 and 44 are beginning to separate. It can be seen that the second tip portion 44 is partially expanded about the first tip portion 42. Additionally as shown in FIG. 5B, a second guidewire 102 is extended from a lumen of the first guidewire 100.

Referring now to FIG. 5C there is shown a cross-sectional view of the composite tip assembly 40 in accordance with the present invention wherein the two tip portions 42 and 44 are almost completely separated and wherein the second guidewire 102 has been extended from a lumen of the first guidewire 100.

Referring now to FIG. 5D there is shown an end view of the composite tip assembly 40 in accordance with the present invention wherein the two tip portions 42 and 44 have been completely separated from each other to form two distinct tips. Additionally, as shown, the first guidewire 100 and the second guidewire 102 are each captured within a tip portion 42 and 44 respectively. Additionally, it can be seen that the first and second tip portions 42 and 44 resiliently close about the respective guidewire disposed therethrough, thus enabling the guidewire to be used as a guide for the tip portions 42 and 44.

It shall be understood that tip assembly 40 as shown herein and described above is an exemplary embodiment of a tip assembly. It is contemplated that various modifications may be made to the geometry and/or the material selection of the tip in order to enable similar functionality as described above. In accordance with an alternative embodiment, the tip assembly 40 may comprise at least two tip portions which are disposed adjacent to one another in longitudinal alignment, wherein the two tip portions may or may not be detachably coupled to each other.

Methods of Use

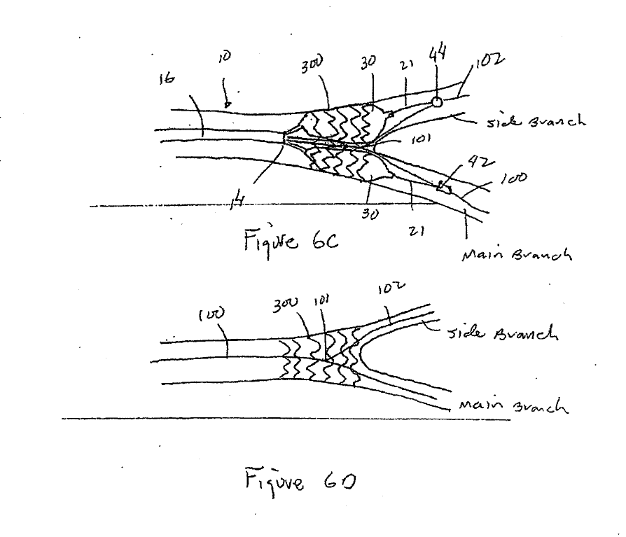

Referring now to FIGS. 6A through 6D there is shown an exemplary embodiment of a method of use of the catheter 10 in accordance with the present invention to deploy an endoprosthesis at a bifurcation location or adjacent to a bifurcation location. p Referring now to FIG. 6A there is shown the distal tip portion of the catheter 10 in accordance with the present invention. As shown in FIG. 6A an expandable medical device 300 is shown disposed radially about the inflatable members 30. A guidewire 100 is shown disposed through at least a portion of guidewire lumen of the catheter shaft and through the tip assembly 40. As shown, the distal end of the catheter 10 is placed adjacent to a bifurcation in a lumen such as a vessel or artery.

Referring now to FIG. 6B there is shown the distal tip of the catheter 10 in accordance with the present invention, wherein the tip assembly 40 has been separated into two tips 42 and 44. Additionally as shown in FIG. 6B and described above, a second guidewire 102 is advanced from a portion 101 of the first guidewire 100. Each of the tips 42 and 44 are guided to a desired location by their respective guidewires.

Referring now to FIG. 6C the distal end of the catheter 10 has been advanced further into the lumen, wherein each of the guidewires respectively guides tips 42 and 44 along the main branch of the lumen and along the side branch of the lumen. Additionally, as shown in FIG. 6C, the inflatable members 30 have been inflated after the expandable endoprosthesis has been properly positioned with respect to the geometry of the main branch and the side branch of the lumen.

Referring now to FIG. 6D there is shown the expanded endoprosthesis 300 as placed in the lumen adjacent the side branch, wherein the catheter 10 has been removed but the guidewires 100 and 102 remain in position in the lumen. An advantage of the present system over previous systems is the present system allows for the placement of an expandable endoprosthesis as shown and described above while maintaining guidewires in the main branch and the side branch of the vessel. If desired, additional medical devices may then be placed over the guidewire to perform additional medical procedures. Further still, in the event of plaque shift or rupture in either the main branch or of the side branch of the vessel, by having the guidewire remain in the respective branch during the procedure enables a user to re-access the site easily.

An additional advantage of the present system over previous systems is the present system is capable of working in conjunction with a coaxial guidewire, which allows for placement of a guidewire within each of the branch vessels without creating twists between the guidewires.

In accordance with the present invention, it is contemplated that although the present invention was described in use with a guidewire 100, wherein the guidewire 100 is a coaxial guidewire having an opening 101 and a second guidewire 102 disposed slidably therein, it is contemplated that the catheter 10 as shown and described and the method shown and described herein may be completed by utilizing two separate guidewires.

### Alternate Embodiment

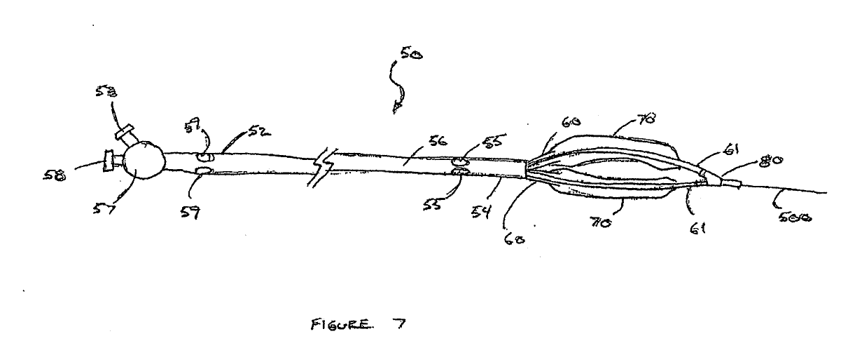

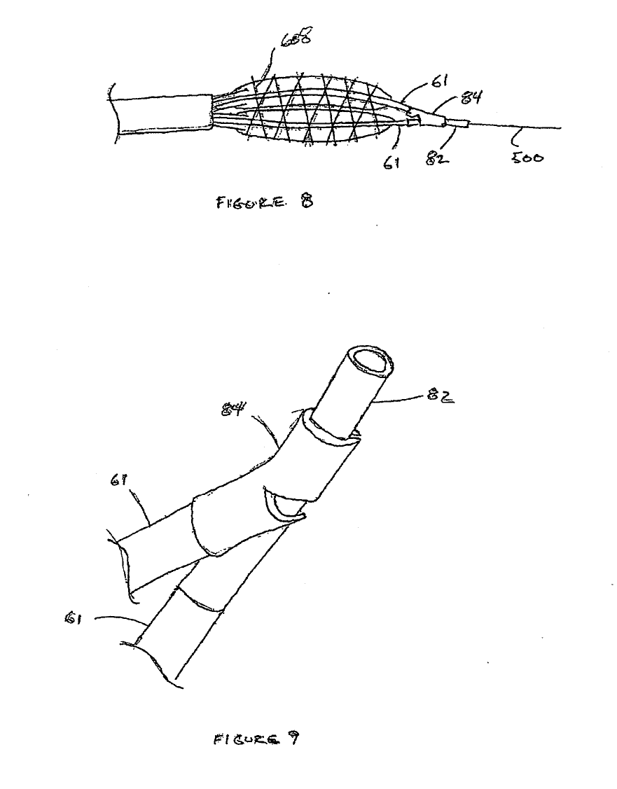

In accordance with the present invention an alternative embodiment of a delivery catheter will be described in detail with reference to FIGS. 7-11D. In accordance with the alternative embodiment of the present invention there is provided a catheter, wherein the catheter is configured to deliver and deploy at least one expandable member within a lumen. The catheter includes a first end, a second end and a shaft extending therebetween At least one inflatable member is disposed adjacent the distal end and is coupled to an inflation lumen extending through the shaft of the catheter, preferably at least two inflatable members are disposed adjacent the distal end, wherein each of the inflatable members includes a separate inflation lumen, thereby allowing independent inflation of the inflatable members.

In use, the catheter is delivered within a lumen by tracking at least a portion of the catheter over a first guidewire. The distal end is tracked to a location adjacent a bifurcation within a lumen to be treated. A second guidewire is advanced through at least a portion of the catheter to a point distal to the inflatable members, at which point, the distal tip of the catheter separates from one composite tip to two independent tips. Preferably, the distal tips each become associated with a respective guidewire and are tracked to a location adjacent a bifurcation to be treated, where an endoprosthesis can then be deployed in response to inflation of the inflatable members.

The delivery system in accordance with the alternative embodiment differs from that described above, in that the guidewire lumens disposed through each of the inflatable members extends distally through the inflatable members and terminates at the distal tip location, wherein the catheter in accordance with the alternative embodiment can be delivered to a bifurcation site over a single guidewire, wherein a second guidewire can then be deployed into the branch lumen of the bifurcation. Unlike the delivery system described above, the delivery system of the alternative embodiment does not require the use of a special guidewire in order to place the system within the bifurcation.

Referring now to FIG. 7 there is shown an exemplary embodiment of the alternative embodiment of a catheter in accordance with the present invention. As shown in FIG. 7, the catheter 50 has a proximal end 52, a distal end 54 and a shaft 56 extending therebetween. A luer fitting 57 is disposed adjacent the proximal end 52 of the shaft 56. As shown in FIG. 7, the luer fitting includes an aperture 58 disposed therethrough, wherein the aperture 58 is in fluid communication with at least one lumen 51 (not shown) extending along the length of the shaft 56. Lumen 51 is configured to receive at least one guidewire therethrough as will be described in greater detail below. The luer 57 may further include an inflation fitting 53, wherein the inflation fitting is in fluid communication with at least one inflation lumen 60 which extends along the length of the shaft 56, wherein the inflation lumen 60 is in fluid communication with at least one inflatable member 70 disposed adjacent the distal end 54 of the shaft 56 as will be described in detail below.

Disposed adjacent the distal end 54 of the shaft 56 is at least one inflatable member 70 as shown in FIG. 7 and described above. Preferably, the catheter 50 includes at least two inflatable members 70 disposed adjacent the distal end 54 of the shaft 56. The inflatable members 70 are coupled to the shaft 56 through inflation lumens 60, wherein the inflation lumens 60 extend along the length of the shaft 56 and are fluidly coupled to the inflation port 53 disposed on the luer 57. The inflatable members 70 are in fluid communication with each respective inflation lumen, thereby being capable of being expanded from a first diameter to a second diameter. The distal guidewire lumens 61 extend beyond a distal end of each of the inflatable members and terminate at a tip assembly 80.

As further shown in FIG. 7, the catheter 50 may be configured to receive a guidewire along its entire length within each of the guidewire lumens 61 disposed within the shaft 56. Alternatively, as shown in FIG. 7, the catheter 50 may include at least one port 55 formed within the wall of the shaft 56 and in communication with the guidewire lumen 61, wherein a guidewire may be placed within the port 55 and extended beyond the distal end of the shaft 56, as shown. Such a configuration is commonly referred to as a rapid-exchange catheter. It is further contemplated, that the catheter 50 may include a second port 59 adjacent the proximal end 52 of the shaft 56, wherein the second port 59 is associated with a guidewire lumen 61, wherein a guidewire 500 may be disposed through the second port 59 and along the length of the shaft and through tip assembly 80 of the catheter 50. By allowing the guidewire to pass through the second port 59 and not through the aperture 58 of the luer 57, the aperture 58 may be utilized for other functions, such as a second inflation lumen.

Referring now to FIG. 8, there is shown an exemplary embodiment of the distal tip of the catheter 50 in accordance with the present invention, wherein an endoprosthesis 600 such as a stent is shown radially disposed about the first and second inflatable members 70.

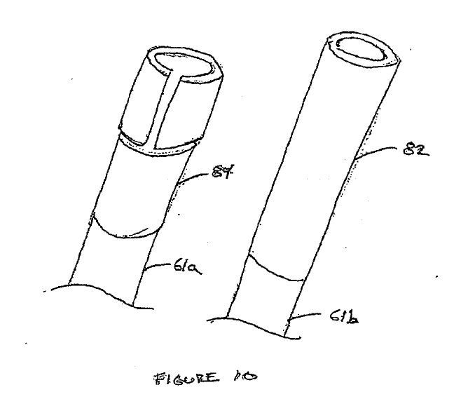

Referring now to FIG. 9, there is shown an exemplary embodiment of a tip member 80 in accordance with the present invention. As shown in FIG. 9, the tip assembly 80 is a composite tip comprising a first tip member 82 and a second tip member 84, wherein the first and second tip members maybe detachably coupled together to form the composite tip assembly 80 as shown. Tip members 82 and 84 are fixedly attached to the distal ends of each of the guidewire lumens 61a and 61b respectively.

Referring now to FIG. 10 there is shown the tip assembly 80, wherein the first and second tip members 82 and 84 have been separated into two distinct tips each having a guidewire lumen 61a and 61b disposed therethrough respectively. Each of the first and second tip members are fixedly attached to their respective guidewire lumens 61a and 61b, wherein when separated as shown in FIG. 10, the catheter 50 converts from a single tipped catheter to a dual tip catheter as will be described below with regard to the methods in accordance with the present invention.

The body of tip member 82 is preferably constructed having a generally cylindrical profile, wherein its length is at least as long as tip member 84 to act as the leading tip while tracking over the guidewire. The body of tip member 84 includes a slot that is disposed extending along a longitudinal length of the body, and a slot that is disposed extending along a radial length of the body, the radial slot beginning at the termination of the longitudinal slot and extending in both radial directions, wherein the slots enable the bodies to expand in diameter, thereby allowing the body of the tip member 82 to nest within the tip member 84 as shown in FIG. 9. The tip members 82 and 84 are preferably constructed of a resilient pliable material, wherein the bodies are capable of flexing and/or expanding about one another to nest together as shown in FIG. 9. Additionally, the tip members 82 and 84 must be constructed of a material which has a sufficiently low durometer such as to not cause damage to a lumen in which the catheter is disposed therein.

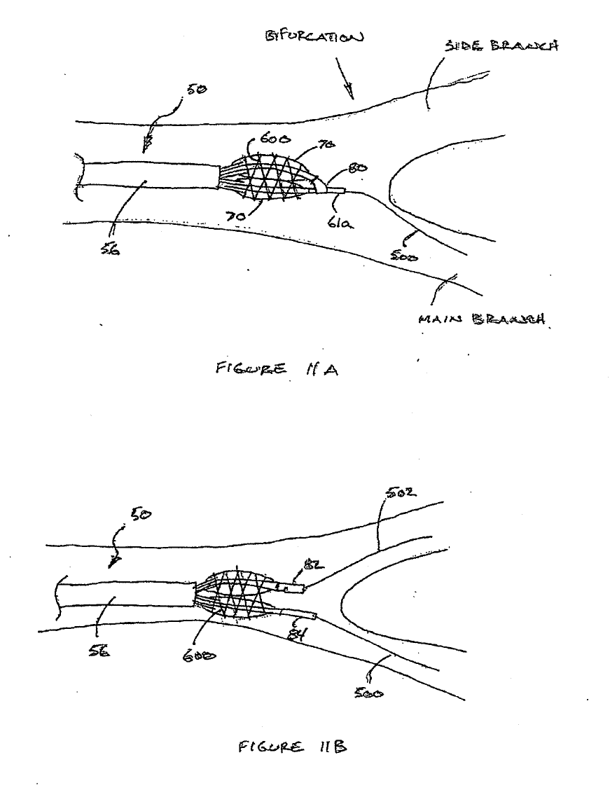

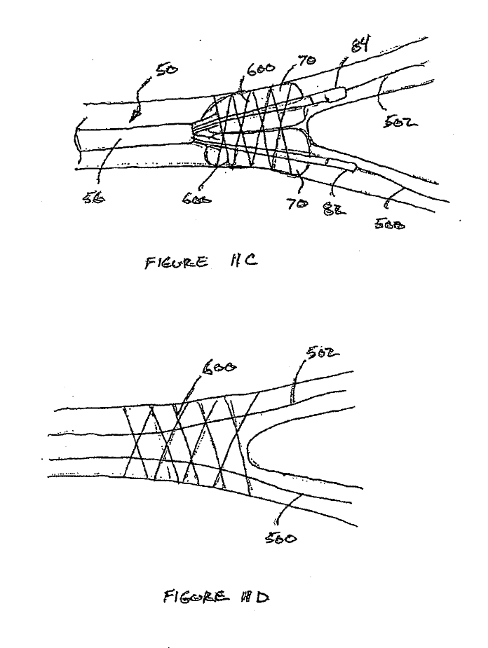

Referring now to FIGS. 11A through 11D there is shown an exemplary embodiment of a method of use of the catheter 50 in accordance with the present invention to deploy an endoprosthesis at a bifurcation location or adjacent to a bifurcation location.

Referring now to FIG. 11A there is shown the distal tip portion of the catheter 50 in accordance with the present invention. As shown in FIG. 11A an expandable endoprosthesis 600 is shown disposed radially about the inflatable members 70. A guidewire 500 is shown disposed through guidewire lumen 61 a of the catheter shaft and through the tip assembly 80. As shown, the distal end of the catheter 50 is placed adjacent to a bifurcation in a lumen such as a vessel or artery.

Referring now to FIG. 11B there is shown the distal tip of the catheter 50 in accordance with the present invention, wherein the tip assembly 80 has been separated into two tips 82 and 84. Additionally as shown in FIG. 11B and described above, a second guidewire 502 is advanced through guidewire lumen 61b from a point proximal to the inflatable member. After reaching the tip assembly 80, the second guidewire forces tip member 84 to increase in diameter, thereby decoupling tip member 84 from tip member 82. After becoming decoupled, tip member 84 then relaxes, leaving the second guidewire 502 nesting within the tip member 84. Each of the tips 82 and 84 are guided to a desired location by their respective guidewires.

Referring now to FIG. 11C the distal end of the catheter 50 has been advanced further into the lumen, wherein each of the guidewires respectively guides tips 82 and 84 along the main branch of the lumen and along the side branch of the lumen. Additionally, as shown in FIG. 11C, the inflatable members 70 have been inflated after the expandable endoprosthesis 600 has been properly positioned with respect to the geometry of the main branch and the side branch of the lumen.

Referring now to FIG. 11D there is shown the expanded endoprosthesis 600 as placed in the lumen adjacent the side branch, wherein the catheter 50 has been removed but the guidewires 500 and 502 remain in position in the lumen. An advantage of the present system over previous systems is the present system allows for the placement of an expandable endoprosthesis as shown and described above while maintaining guidewires in the main branch and the side branch of the vessel. If desired, additional medical devices may then be placed over the guidewires to perform additional medical procedures. Further still, in the event of plaque shift or rupture in either the main branch or the side branch of the vessel, by having the guidewires remain in the respective branch during the procedure enables a user to re-access the site easily.

The invention is susceptible to various modifications and alternative forms, and specific examples thereof have been shown by way of example in the drawings and are herein described in detail. It should be understood, however, that the invention is not to be limited to the particular forms or methods disclosed, but to the contrary, the invention is to cover all modifications, equivalents, and alternatives falling within the spirit and scope of the claims.

What is claimed is:

1. A stent delivery system for delivering and deploying a radially expandable stent at a strategic orientation and location in a body vessel, said delivery system comprising:
an elongated shaft,
a first inflatable member extending from a distal end of the elongated shaft;
a second inflatable member extending from the distal end of the elongated shaft and disposed adjacent to the first inflatable member, wherein the first and second inflatable members are in fluid communication with each other, and
a tip assembly, the tip assembly including a first tip portion associated with the first inflatable member and a second tip portion associated with the second inflatable member, wherein the first and second tip portions are configured to be detachably associated with each other.

2. The stent delivery system according to claim 1, further including an expandable endoprosthesis radially disposed about the first and second inflatable members.

3. A method of placing an expandable endoprosthesis in a lumen, comprising:
advancing a endoprosthesis delivery system over at least one guidewire to a location adjacent a bifurcation location, the guidewire extending through a tip assembly;
advancing a second guidewire through the tip assembly;
separating the tip assembly to form a first tip and a second tip, wherein the first tip is disposed about one of the guidewires and the second tip is disposed about the other guidewire;
advancing the delivery system to the bifurcation location;
deploying the endoprosthesis.
